(19) United States
(12) Patent Application Publication
(10) Pub. No.: US 2026/0174656 A1
(43) Pub. Date: Jun. 25, 2026

(54) COMPOSITIONS AND METHODS AND USES THERETO

(71) Applicant: Innospec Limited, Ellesmere Port (GB)

(72) Inventors: Anh VU THI NGOC, Bratislava (SK); Peter CLARK, Ellesmere Port (GB)

(21) Appl. No.: 19/123,160

(22) PCT Filed: Oct. 23, 2023
(86) PCT No.: PCT/GB2023/052776

§ 371 Date: Apr. 22, 2025

(30) Foreign Application Priority Data
Oct. 24, 2022 (GB) 2215745.7

(51) Int. Cl. A61K 8/46; A61K 8/34; A61K 8/368; A61K 8/44; A61K 8/60; A61Q 5/02; A61Q 19/10

(52) U.S. Cl. CPC A61K 8/466; A61K 8/34; A61K 8/368; A61K 8/44; A61K 8/602; A61Q 5/02; A61Q 19/10; A61K 2800/87

(57) ABSTRACT

A personal care concentrate composition including an anionic surfactant: an amphoteric surfactant; and a non-ionic surfactant in which the viscosity of the composition increases upon dilution with water.

The present invention relates to personal care compositions. In particular, the invention relates to personal care concentrate compositions which are diluted prior to use.

Due to environmental concerns there is a continuing drive to reduce the consumption of single-use plastic packaging. It is therefore desirable to provide products in packaging which can be re-used and/or to provide products which require less packaging.

Soap boars, shampoo bars and synthetic detergent bars are all known. However, consumers are attracted to liquid shampoo and body wash compositions and desire the product consistency that they are used to.

Formulated ready-to-use personal care products such as shampoos and body washes typically contain up to 80% water. Thus the transport of these compositions involves transport of large volumes of water.

It is therefore desirable to provide personal care compositions having a reduced water content.

There have been some previous attempts to provide concentrated personal care products to be formulated by the end user. However, it has proved difficult to provide a product that is easy to handle and creates a homogenous, clear and stable product with the desired rheology, properties and feel of ready-to-use products. Previous products have, for example, required the user to carry out significant mixing steps and sometimes required heating to provide the formulated composition. Other compositions have required the use of a specific dispenser, increasing the packaging used and the complexity of the product. Also, conventional surfactant concentrates often require salts (e.g. NaCl) or polymeric viscosifiers to be added in order to achieve the desired viscosity in ready-to-use personal care products.

A further problem with concentrate compositions of the prior art is that they typically have high viscosities and thus do not flow easily. Such compositions can be difficult to pump and large amounts of energy are used in their production.

It is the aim of the present invention to provide a concentrated personal care composition which has a low viscosity and that can be easily diluted by a user to provide a stable, thickened product.

According to a first aspect of the present invention there is provided a personal care concentrate composition comprising:
(a) an anionic surfactant;
(b) an amphoteric surfactant;
(c) a non-ionic surfactant;

wherein the viscosity of the composition increases upon dilution with water.

The first aspect of the present invention relates to a personal care concentrate composition. For the avoidance of doubt, by a concentrate composition we mean to refer to a composition comprising active ingredients in a high concentration, with a reduced volume of water. The concentrate composition is intended to be diluted prior to use.

The personal care concentrate compositions of the present invention comprise (a) an anionic surfactant, (b) an amphoteric surfactant and (c) a non-ionic surfactant. The personal care concentrate composition may comprise a mixture of two or more anionic surfactants and/or two or more amphoteric surfactants and/or two or more non-ionic surfactants.

For the avoidance of doubt references herein to “an” or “the” anionic surfactant, “an” or “the” amphoteric surfactant or “an” or “the” non-ionic surfactant include embodiments in which mixtures of such surfactants are present.

The personal care concentrate comprises (a) an anionic surfactant.

Any suitable anionic surfactant may be included. Such compounds will be known to the person skilled in the art.

Suitable anionic surfactants for use herein include salts of: fatty acids; alkoxylated carboxylic acids; ester carboxylates; ethoxylated ester carboxylates; mono- or dialkyl sulfates; mono- or dialkyl ether sulfates; lauryl ether sulfates; alkyl sulfonates; alkyl aryl sulfonates; primary alkane disulfonates; alkene sulfonates; hydroxyalkane sulfonates; isethionates, alkyl isethionates, acyl isethionates, acyl alkyl isethionates, alkyl glyceryl ethersulfonates; alpha-olefin sulfonates; alkyl phosphates; sulfonates of alkylphenolpolyglycol ethers; alkyl sulfopolycarboxylic acid esters; alkyl sulfosuccinates; alkyl ether sulfosuccinates; taurates; acyl taurates; acyl N-alkyl taurates; products of condensation of fatty acids with oxy- and aminoalkanesulfonic acids; sulfated derivatives of fatty acids and polyglycols; alkyl and acyl sarcosinates; sulfoacetates; alkyl phosphates; alkyl phosphate esters; acyl lactates; alkanolamides of sulfated fatty acids, lipoamino acids and acyl substituted amino acids, for example acyl glycinates and acyl glutamates. Particularly exemplary salts, where applicable, are the sodium, potassium, ammonium, magnesium and triethanolamine salts.

Suitable anionic surfactants for use in the present invention typically comprise one or more moieties selected from carbonate, phosphate, phosphonate, sulphate, sulphonate, carboxylate and mixtures thereof.

Preferred anionic surfactants include alkyl or alkenyl sulfates, alkyl or alkenyl ether sulfates, alkyl glyceryl ether sulfates, taurates, acyl taurates, (alkyl) isethionates (alkyl) acyl isethionates, acyl substituted amino acids, sarcosinates, sulfosuccinates, sulfosuccinamates, sulphoacetates, monoalkyl phosphate esters, di-alkyl phosphate esters, mono-alkyl ether phosphate esters, di-alkyl ether phosphate esters, alpha-olefin sulfonates, acyl lactates, alkyl ether carboxylates, glyceryl ether carboxylates.

Particularly preferred anionic surfactants for use herein include alkyl or alkenyl sulfates, alkyl or alkenyl ether sulfates, taurates, isethionates, acyl substituted amino acids, sarcosinates, and sulfosuccinates.

Illustrative examples of preferred anionic surfactants include sodium lauryl sulphate, sodium lauryl ether sulfate, sodium lauroyl methyl taurate, sodium methyl cocoyl taurate, sodium methyl oleoyl taurate, sodium cocoyl isethionate, sodium cocoyl methyl isethionate, sodium lauroyl isethionate, sodium lauroyl methyl isethionate, sodium lauroyl glycinate, sodium cocoyl glycinate, sodium lauryl sarcosinate, and disodium oleamido mipa sulfosuccinate.

Suitable alkyl or alkenyl sulfates and ether sulfates include compounds of formula:

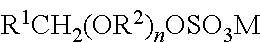

wherein R¹ is a C₅ to C₃₉ alkyl or alkenyl group, R² is an alkylene group having 1 to 4 carbon atoms and n is from 0 to 12.

R² is preferably a propylene or especially an ethylene moiety. Preferably n is from 0 to 6, preferably from 0 to 4, for example from 0 to 2. In some embodiments n is 0. In some embodiments n is 2.

Suitable anionic surfactants include salts of alkyl sulfates, alkyl ether sulfates, fatty acids, carboxylates, alkyl sulfonates, aryl sulfonates, alkyl benzene sulphonates, isethionates, alkyl phosphates, sulfosuccinates, taurates, sarcosinates, sulfoacetates, lactates, acyl amino acids and phosphonates.

Preferably component (a) comprises at least one acyl alkyl isethionate surfactant of formula (I):

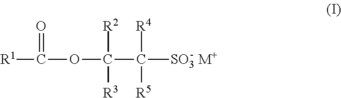

wherein R¹ represents an optionally substituted C₄-C₃₆ hydrocarbyl group;

each of R², R³, R⁴ and R⁵ independently represents hydrogen or a C₁-C₄ alkyl group and wherein at least one of R², R³, R⁴ and R⁵ is not hydrogen; and M⁺ represents a cation.

Component (a) may comprise a mixture of two or more anionic surfactants including a mixture of two more compounds of formula (1).

In the formula (1), R¹ represents an optionally substituted C₄-C₃₆ hydrocarbyl group, R², R³, R⁴ and R⁵ each independently represents hydrogen or a substituted or unsubstituted C₁-C₄ alkyl group, provided that at least one of R², R³, R⁴ and R⁵ is not hydrogen, and M⁺ represents a cation.

Suitably, R¹ represents an optionally substituted C₄-C₃₆ alkyl, C₄-C₃₆ alkenyl, C₆-C₁₂ aryl or C₈-C₂₂ alkyl-C₆-C₁₂ aryl group. More suitably, R¹ represents an optionally substituted C₄-C₃₆ alkyl or C₄-C₃₆ alkenyl group, especially an optionally substituted C₄-C₃₆ alkyl group. Most suitably, R¹ represents a C₄-C₃₆ alkyl or C₄-C₃₆ alkenyl group, especially a C₄-C₃₆ alkyl group.

Suitably, R¹ represents an optionally substituted C₄-C₃₆ alkyl or C₄-C₃₆ alkenyl group, such as an optionally substituted C₈-C₁₈ alkyl or C₈-C₁₈ alkenyl group.

Suitably, R¹ represents a C₄-C₃₆ alkyl or C₄-C₃₆ alkenyl group, such as a C₈-C₁₈ alkyl or C₈-C₁₈ alkenyl group.

Suitably, R¹ represents an optionally substituted C₅-C₃₀ alkyl group, such as an optionally substituted C₇-C₂₄ alkyl group, for example an optionally substituted C₇-C₂₁ alkyl group, preferably an optionally substituted C₇-C₇ alkyl group.

Suitably, R¹ represents a C₅-C₃₀ alkyl group, such as a C₇-C₂₄ alkyl group, for example a C₇-C₂₁ alkyl group, preferably a C₇-C₁₇alkyl group.

R¹ is suitably the residue of a fatty acid. Fatty acids obtained from natural oils often include mixtures of fatty acids. For example, the fatty acid obtained from coconut oil contains a mixture of fatty acids including C₁₂ lauric acid, C₁₄ myristic acid, C₁₆ palmitic acid, C₈ caprylic acid, C₁₀ capric acid and C₁₈ stearic and oleic acid.

R¹ may include the residue of one or more naturally occurring fatty acids and/or of one or more synthetic fatty acids. For example, R¹ may consist essentially of the residue of a single fatty acid.

Examples of carboxylic acids from which R¹ may be derived include coco acid, hexanoic acid, caproic acid, caprylic acid, capric acid, lauric acid, myristic acid, palmitic acid, palmitoleic acid, stearic acid, oleic acid, linoleic acid, arachidic acid, gadoleic acid, arachidonic acid, eicosapentanoic acid, behinic acid, erucic acid, docosahexanoic lignoceric acid, naturally occurring fatty acids such as those obtained from coconut oil, tallow, palm kernel oil, butterfat, palm oil, olive oil, corn oil, linseed oil, peanut oil, fish oil and rapeseed oil; synthetic fatty acids made as chains of a single length or a selected distribution of chain lengths; and mixtures thereof. Suitably R¹ comprises the residue of coco acid, the residue of mixed fatty acids derived from coconut oil or the residue of mixed fatty acids derived from palm kernel oil. More suitably, R¹ predominantly comprises the residue of a saturated fatty acid having 12 carbon atoms.

The acyl alkyl isethionate surfactant of the formula (1) may be prepared by any of the methods disclosed in the prior art, for example see the methods described in WO94/09763 and WO2005/075623.

In some embodiments only a single acyl alkyl isethionate surfactant of the formula (1) may be present in the concentrate composition of the first aspect. In some embodiments a mixture of two or more acyl alkyl isethionate surfactants of the formula (1) may be present. In such embodiments the above amounts refer to the total amounts of all acyl alkyl isethionate surfactants of the formula (1) present in the composition.

When any of R², R³, R⁴ and R⁵ represents an optionally substituted C₁-C₄ alkyl group, the alkyl group is suitably butyl, n-propyl, ethyl or methyl, such as ethyl or methyl, most preferably methyl.

Preferably one of the groups R², R³, R⁴ and R⁵ represents an optionally substituted C₁-C₄ alkyl group and the remaining groups represent hydrogen. For example, R² may represent an optionally substituted C₁-C₄ alkyl group and R³, R⁴ and R⁵ may all represent hydrogen. For example, R⁴ may represent an optionally substituted C₁-C₄ alkyl group and R², R³ and R⁵ may all represent hydrogen.

Preferably, R² represents a C₁-C₄ alkyl group and R³, R⁴ and R⁵ all represent hydrogen. Preferably, R⁴ represents a C₁-C₄ alkyl group and R², R³ and R⁵ all represent hydrogen. Most preferably, R² represents a methyl group and R³, R⁴ and R⁵ all represent hydrogen. Most preferably, R⁴ represents a methyl group and R², R³ and R⁵ all represent hydrogen.

Suitably, M⁺ represents a metal cation or an optionally substituted ammonium cation, preferably a metal cation. By “optionally substituted ammonium cation”, we mean to refer to an ammonium cation wherein the nitrogen atom may be substituted with from 1 to 4 optionally substituted hydrocarbyl groups. Suitable ammonium cations include those derived from alkyl amines and alkanolamines. Preferred ammonium cations include isopropanolamine, isopropylamine, ethanolamine, diethanolamine, triethanolamine and 2-amino-2-methyl-1,3-propanediol (AMPD). Preferred ammonium cations include NH₄⁺ and the ammonium cation of triethanolamine.

Suitable metal cations include alkali metal cations, for example sodium, lithium and potassium cations, and alkaline earth metal cations, for example calcium and magnesium cations. Suitably, M⁺ represents an alkali metal cation or an optionally substituted ammonium cation. Preferably, M⁺ represents a zinc, potassium or sodium cation. Most preferably, M⁺ represents a sodium cation.

The skilled person will appreciate that when M⁺ is a divalent metal cation two moles of anion will be present for each mole of cation.

Preferably component (a) comprises an acyl alkyl isethionate surfactant of formula (1) selected from one or more of sodium lauroyl methyl isethionate, sodium cocoyl methyl isethionate and sodium oleoyl methyl isethionate. Sodium lauroyl methyl isethionate (SLMI) is especially preferred.

The acyl alkyl isethionate surfactant of formula (1) may comprise the reaction product of sodium methyl isethionate and a fatty acid, that is a compound of formula R¹COOCHR²CHR⁴SO₃·M⁺ in which one of R² and R⁴ is methyl and the other is hydrogen. In some embodiments, mixtures of these isomers are present.

Suitably such mixtures comprise approximately 90% of compounds in which R² is methyl and R⁴ is hydrogen and approximately 10% of compounds in which R² is hydrogen and R⁴ is methyl.

The anionic surfactant (a) is suitably present in the concentrate composition in an amount of from 0.1 to 50 wt %, preferably from 1 to 40 wt %, more preferably from 2 to 30 wt %, preferably from 3 to 25 wt %, preferably from 5 to 20 wt %, more preferably from 7 to 18 wt %, for example from 10 to 15 wt %.

The above amounts refer to the amount of active surfactant present in the concentrate composition. The skilled person will appreciate that commercial sources of surfactant compositions often comprise unreacted starting materials, by-products, impurities and/or solvents.

In embodiments in which the concentrate composition comprises two or more anionic surfactants the above amounts refer to the total amounts of all such surfactants present in the composition.

The personal care concentrate composition of the present invention further comprises (b) an amphoteric surfactant.

Suitable amphoteric surfactants for use in compositions of the first aspect of the invention include those based on fatty nitrogen derivatives and those based on betaines.

Suitable amphoteric or zwitterionic surfactants may be selected from betaines, for example alkyl betaines, alkylamidopropyl betaines, for example cocamidopropyl betaine, alkylamidopropyl sultaines alkylamidopropyl hydroxy sultaines, alkylamphoacetates, alkylamphodiacetates, alkyl propionates, alkylamphodipropionates, alkylamphopropionates, alkyliminodipropionates and alkyliminodiacetate.

Suitable amphoteric or zwitterionic surfactants for use in compositions of the first aspect may include those which have an alkyl or alkenyl group of 7 to 22 carbon atoms and comply with an overall structural formula:

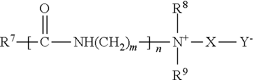

where R⁷ is alkyl or alkenyl of 7 to 22 carbon atoms; R⁸ and R⁹ are each independently alkyl, hydroxyalkyl or carboxyalkyl of 1 to 6 carbon atoms; m is 2 to 4; n is 0 or 1; X is alkylene of 1 to 6 carbon atoms optionally substituted with hydroxyl; and Y is —CO₂ or —SO₃.

Amphoteric or zwitterionic surfactants may include simple betaines of formula:

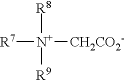

and amido betaines of formula:

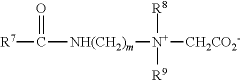

where m is 2 or 3.

In both formulae R⁷, R⁸ and R⁹ are as defined previously. R⁷ may, in particular, be a mixture of C₁₂ and C₁₄ alkyl groups derived from coconut so that at least half, preferably at least three quarters, of the groups R⁷ has 10 to 14 carbon atoms. R⁸ and R⁹ are preferably methyl.

Amphoteric or zwitterionic surfactants may include sulfobetaines of formula:

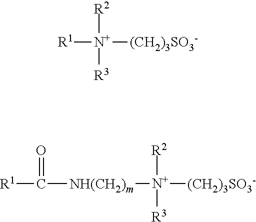

where m is 2 or 3, or variants of these in which —(CH₂)₃SO₃⁻ is replaced by

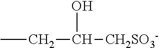

where R⁷, R⁸ and R⁹ in these formulae are as defined previously.

Amphoteric or zwitterionic surfactants may include amphoacetates and diamphoacetates.

Amphoacetates generally conform to the following formula:

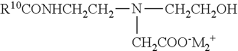

Diamphoacetates generally conform to the following formula:

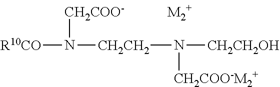

where R¹⁰ is an aliphatic group of 8 to 22 carbon atoms and M₂⁺ is a cation such as sodium, potassium, ammonium, or substituted ammonium.

Suitable acetate-based surfactants include lauroamphoacetate; alkyl amphoacetate; sodium alkyl amphoacetate; cocoampho(di)acetate; cocoamphoacetate; disodium cocoamphodiacetate; sodium cocoamphoacetate; disodium cocoamphodiacetate; disodium capryloamphodiacete; disodium lauroamphoacetate; sodium lauroamphoacetate and disodium wheatgermamphodiacetate.

Suitable betaine surfactants include alkylamido betaine; alkyl betaine, C_{12/14} alkyldimethyl betaine; cocoamidopropylbetaine; tallow bis(hydroxyethyl) betaine; hexadecyldimethylbetaine; cocodimethylbetaine; alkyl amido propyl sulfo betaine; alkyl dimethyl amine betaine; coco amido propyl dimethyl betaine; alkyl amido propyl dimethyl amine betaine; cocamidopropyl betaine; lauryl betaine; laurylamidopropl betaine, coco amido betaine, lauryl amido betaine, alkyl amino betaine; alkyl amido betaine; coco betaine; lauryl betaine; diemethicone propyl PG-betaine; oleyl betaine; N-alkyldimethyl betaine; coco biguamide derivative, C₈ amido betaine; C₁₂ amido betaine; lauryl dimethyl betaine; alkylamide propyl betaine; amido betaine; alkyl betaine; cetyl betaine; oleamidopropyl betaine; isostearamidopropyl betaine; lauramidopropyl betaine; 2-alkyl-N-carboxymethyl-N-hydroxyethyl imidazolinium betaine; 2-alkyl-N-carboxyethyl-N-hydroxyethyl imidazolinium betaine; 2-alkyl-N-sodium carboxymethyl-N-carboxymethyl oxyethyl imidazolinium betaine; N-alkyl acid amidopropyl-N,N-dimethyl-N-(3-sulfopropyl)-ammonium-betaine; N-alkyl-N,N-dimethyl-N-(3-sulfopropyl)-ammonium-betaine; cocodimethyl betaine; apricotamidopropyl betaine; isostearamidopropyl betaine; myristamidopropyl betaine; palmitamidopropyl betaine; alkamidopropyl hydroxyl sultaine; cocamidopropyl hydroxyl sultaine; undecylenamidopropyl betaine; cocoamidosulfobetaine; alkyl amido betaine; C_{12/18} alkyl amido propyl dimethyl amine betaine; lauryldimethyl betaine; ricinol amidobetaine; tallow aminobetaine.

Suitable glycinate surfactants include acyl glycinates such as cocoamphocarboxyglycinate; tallowamphocarboxygycinate; capryloamphocarboxyglycinate, oleoamphocarboxyglycinate, bis-2-hydroxyethyl tallow glycinate; lauryl amphoglycinate; tallow polyamphoglycinate; coco amphoglycinate; oleic polyamphoglycinate; N—C_{10/12} fatty acid amidoethyl-N-(2-hydroxyethyl)-glycinate; N—C_{12/18}-fatty acid amidoethyl-N-(2-hydroxyethyl)-glycinate; dihydroxyethyl tallow gycinate.

Preferred acetate-based amphoteric surfactants for use herein include sodium lauroamphoacetate, disodium lauroamphoacetate and mixtures thereof.

Preferred betaine surfactants for use herein include cocoamidopropyl betaine.

Preferred sultaine surfactants for use herein include cocoamidopropylhydroxy sultaine.

Preferably component (b) comprises cocoamidopropyl betaine.

The amphoteric surfactant (b) is suitably present in the concentrate composition in an amount of from 0.1 to 50 wt %, preferably from 1 to 40 wt %, more preferably from 2 to 30 wt %, preferably from 3 to 25 wt %, preferably from 5 to 20 wt %, more preferably from 7 to 18 wt %, for example from 10 to 15 wt %.

The above amounts refer to the amount of active surfactant present in the concentrate composition. The skilled person will appreciate that commercial sources of surfactant compositions often comprise unreacted starting materials, by-products, impurities and/or solvents.

In embodiments in which the concentrate composition comprises two or more amphoteric surfactants the above amounts refer to the total amounts of all such surfactants present in the composition.

The personal care concentrate composition further comprises (c) a non-ionic surfactant.

Suitable non-ionic surfactants for use in compositions of the first aspect of the invention include alcohol alkoxylates such as alcohol ethoxylates, alcohol propoxylates, and ethylene oxide/propylene oxide copolymer derived surfactants, aliphatic esters, aromatic esters, sugar esters, especially sorbitan esters, alkyl glucosides, fatty acid alkoxylates such as fatty acid ethoxylates and fatty acid propoxylates or polyethylene glycol esters and partial esters, glycerol esters including glycerol partial esters and glycerol triesters, fatty alcohols (such as cetearyl alcohol, lauryl alcohol, stearyl alcohol, behenyl alcohol), alkanolamides and amineoxides.

Suitable non-ionic surfactants may be selected from the following: reaction products of compounds having a hydrophobic group and a reactive hydrogen atom, for example aliphatic alcohols, acids, amides or alkyl phenols with alkylene oxides, especially ethylene oxide either alone or with propylene oxide (for example alkyl(C₆-C₂₂) phenol-ethylene oxide condensates, the condensation products of aliphatic (C₅-C₁₈) primary or secondary linear or branched alcohols with ethylene oxide, and products made by condensation of ethylene oxide with the reaction products of propylene oxide and ethylenediamine); long chain tertiary amine oxides, long chain tertiary phosphine oxides and dialkyl sulfoxides; alkyl amine oxides, alkyl amido amine oxides; alkyl tertiary phosphine oxides; alkoxyl alkyl amines; sorbitan; sorbitan esters; sorbitan ester alkoxylates; glycerol ester alkoxylates; sucrose esters; sugar amides, such as a saccharide amide; lactobionamides; and alkyl saccharide nonionic surfactants, for example alkyl glycosides.

Preferred non-ionic surfactants for use herein include alcohol ethoxylates, amine oxides and alkyl saccharide nonionic surfactants.

Most preferably component (c) comprises an alkyl saccharide compound.

By alkyl saccharide compound we mean to refer to a compound including an alkyl group and a saccharide moiety.

The alkyl group may be bound to the saccharide moiety via a carbon-carbon bond or via a carbon-oxygen bond. Preferably it is bound to the saccharide moiety via a carbon-oxygen bond, for example via an ester linkage or an ether linkage. Most preferably it is bound to the saccharide moiety via an ether linkage.

The alkyl saccharide compound may include one or more alkyl groups. Preferably it comprises one alkyl group. The hydrocarbyl group may be an optionally substituted alkyl, alkenyl or alkynylene group. Preferably the alkyl group is an unsubstituted alkyl group. It may be straight chained or may be branched. Most preferably it is straight chained. Especially preferred hydrocarbyl groups are alkyl groups having from 1 to 36 carbon atoms, preferably 2 to 30 carbon atoms, more preferably from 4 to 24 carbon atoms, suitably from 4 to 20 carbon atoms, preferably from 6 to 16 carbon atoms, for example from 8 to 16 carbon atoms and most preferably from 10 to 14 carbon atoms. Preferred are straight chained alkyl groups having from 6 to 16 carbon atoms.

The saccharide moiety of the alkyl saccharide compound may include from 1 to 30 monosaccharide species. Preferably the saccharide moiety comprises from 1 to 10, suitably from 1 to 5, preferably from 1 to 2 monosaccharide units. Any suitable monosaccharide unit may be included. Preferred saccharides include allose, altrose, glucose, mannose, gulose, idose, galactose and talose.

Mixtures of two or more monosaccharides may be present in the saccharide moiety. Preferably the saccharide moiety comprises glucose. More preferably all of the monosaccharide units present in the saccharide moiety are glucose.

In a preferred embodiment the alkyl saccharide compound is an alkyl glucoside (APG), preferably a monoalkyl-glucoside. Suitably the hydrocarbyl-saccharide compound is a compound of general formula (II):

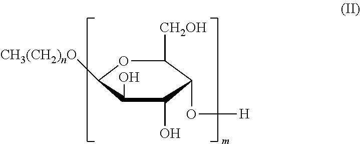

wherein n is from 2 to 24, preferably from 5 to 17, more preferably from 9 to 13 and m is from 1 to 10, preferably from 1 to 2.

Preferably, on average, the hydrocarbyl saccharide compound includes 1 to 1.5, for example about 1.3 glucose units per molecule. As the skilled person will appreciate, a single molecule cannot have 1.3 glucose units. What is meant by an average number of glucose units is the mean number of glucose units per molecule of acylating agent as the sum of all the glucose units present in a sample divided by the total number of molecules of hydrocarbyl saccharide compound.

In preferred embodiments component (c) comprises an alkyl glucoside, preferably lauryl glucoside.

The non-ionic surfactant is suitably present in the concentrate composition in an amount of from 0.1 to 50 wt %, preferably from 1 to 40 wt %, more preferably from 2 to 30 wt %, preferably from 3 to 25 wt %, preferably from 5 to 20 wt %, more preferably from 7 to 18 wt %, for example from 10 to 15 wt %.

The above amounts refer to the amount of active surfactant present in the concentrate composition. The skilled person will appreciate that commercial sources of surfactant compositions often comprise unreacted starting materials, by-products, impurities and/or solvents.

In embodiments in which the concentrate composition comprises two or more non-ionic surfactants the above amounts refer to the total amounts of all such surfactants present in the composition.

The personal care concentrate composition of the present invention comprises a mixture of (a) an anionic surfactant, (b) an amphoteric surfactant and (c) a non-ionic surfactant.

In preferred embodiments the weight ratio of component (a) to component (b) present in the personal care concentrate composition is from 5:1 to 1:5, preferably from 3:1 to 1:3, more preferably from 2:1 to 1:2, most preferably from 1.5:1 to 1:1.5.

In preferred embodiments the weight ratio of component (a) to component (c) present in the personal care concentrate composition is from 5:1 to 1:5, preferably from 3:1 to 1:3, more preferably from 2:1 to 1:2, most preferably from 1.5:1 to 1:1.5.

In preferred embodiments the weight ratio of component (b) to component (c) present in the personal care composition is from 5:1 to 1:5 preferably from 3:1 to 1:3 more preferably from 2:1 to 1:2, most preferably from 1:5 to 1:1.5.

For the avoidance of doubt the above ratios are ratios by weight of active surfactant, ignoring any impurities, byproducts and/or unreacted starting material which may be present.

In an especially preferred embodiment, the personal care concentrate composition of the present invention comprises components (a), (b) and (c) in an approximate 1:1:1 weight ratio. Preferably the weight ratio of component (a) to component (b) to component (c) is 1:1±0.5:1±0.5.

In a preferred embodiment the personal care concentrate composition of the present invention comprises from 5 to 20 wt %, preferably from 10 to 15 wt % component (a); from 5 to 20 wt %, preferably from 10 to 15 wt % component (b); and from 5 to 20 wt %, preferably from 10 to 15 wt % component (c).

Preferably component (a), component (b) and component (c) together comprise at least 20 wt % of the personal care concentrate composition, preferably at least 15 wt %, more preferably at least 20 wt %, suitably at least 25 wt %, preferably at least 30 wt %.

Preferably component (a), component (b) and component (c) together comprise up to 60 wt % of the personal care concentrate composition, preferably up to 50 wt %, more preferably up to 45 wt %, suitably up to 40 wt %.

Preferably component (a), component (b) and component (c) together comprise from 25 to 45 wt % of the personal care concentrate composition, preferably from 30 to 40 wt %, more preferably from 33 to 37 wt %.

The personal care concentrate composition suitably further comprises one or more solvents.

Preferably the personal care concentrate composition is an aqueous composition. In some embodiments the composition may comprise one or more further solvents in addition to water.

Such suitable co-solvents may include polar compounds for example alcohols, glycols and the like.

In preferred embodiments water is the major solvent present in the personal care concentrate composition of the present invention and suitably comprises at least 90 wt % of all solvents present, preferably at least 95 wt %, more preferably at 99 wt %. In especially preferred embodiments water is substantially the only solvent present in the personal care concentrate composition.

The first aspect of the present invention relates to concentrate compositions and not ready to use formulations. An advantage of the concentrate compositions of the present invention is that a reduced volume of water must be transported.

Preferably the personal care concentrate composition of the present invention comprises less than 80 wt % water, preferably less than 75 wt %, more preferably less than 70 wt %.

The personal care concentrate composition may comprise one or more further components commonly used in personal care formulations.

For example the personal care concentrate composition may comprise one or more foaming agents, emollients, dyes, fragrances, chelating agents, thickeners, conditioners, thickening agents, preservatives, antimicrobials, sequestrants, vitamins and derivatives thereof, sunscreens, desquamation actives, anti-wrinkle/anti-atrophy actives, anti-oxidants, skin soothing agents/skin healing agents, skin lightening agents, skin tanning agents, anti-acne medicaments, essential oils, sensates, pigments, pearlescent agents and interference pigments.

Other optional materials can be those materials approved for use in cosmetics and that are described in the CTFA Cosmetic Ingredient Handbook, Second Edition, The Cosmetic, Toiletries, and Fragrance Association, Inc. 1988, 1992.

The personal care concentrate composition of the first aspect preferably comprises a chelating agent. Suitable chelating agents include ethylenediamine-N,N′-disuccinic acid, methylglycinediacetic acid, glutamic acid N,N-diacetic acid, imino disuccinic acid, diethylene triamine pentaacetic acid, ethylenediamine tetraacetic acid, diethylenetriamine penta methylene phosphonic acid, etidronic acid and anions and mixtures thereof.

Preferred chelating agents are biodegradable chelating agents for example ethylenediamine-N,N′-disuccinic acid, methylglycinediacetic acid, glutamic acid N,N-diacetic acid, imino disuccinic acid and anions and mixtures thereof.

An especially preferred chelating agent is the trisodium salt of ethylenediamine-N,N′-disuccinic acid.

The chelating agent is suitably present in an amount of from 0.1 to 2 wt %.

The personal care concentrate composition of the first aspect preferably comprises one or more preservatives and/or antioxidants. Suitable compounds of this type will be known to the person skilled in the art and include, for example phenoxyethanol and sodium benzoate.

In preferred embodiments the personal care concentrate composition comprises from 0.1 to 10 wt %, preferably 0.5 to 5 wt % of one or more preservatives and/or antioxidants.

In some embodiments the personal care concentrate composition comprises from 0.1 to 5 wt %, preferably 1.5 to 2.5 wt % of phenoxyethanol and/or from 0.1 to 5 wt %, preferably 1.5 to 2.5 wt % of sodium benzoate.

The personal care concentrate composition of the present invention may further comprise a pH modifier. Any suitable acid or base may be used as a pH modifier. Preferred pH modifiers are those which are cosmetically acceptable and the selection of an appropriate acid or base would be within the competence of the person skilled in the art. An especially preferred acid is critic acid. An especially preferred base is sodium hydroxide.

Preferably the pH of the personal care concentrate composition is from 4.5 to 8, preferably from 5 to 7, more preferably from 6 to 6.5.

Other optional components may be included in the personal care concentrate composition.

These will depend on the intended use of the final product. The selection of suitable further components will be within the competence of the person skilled in the art.

In one preferred embodiment the personal care concentrate composition of the present invention comprises:
from 5 to 20 wt %, preferably from 10 to 15 wt % of an anionic surfactant;
from 5 to 20 wt %, preferably from 10 to 15 wt % of an amphoteric surfactant;
from 5 to 20 wt %, preferably from 10 to 15 wt % of a non-ionic surfactant;
from 0.1 to 2 wt % of a chelating agent; and
from 0.1 to 10 wt %, preferably from 0.5 to 5 wt % of one or more preservatives and/or antioxidants.

In one preferred embodiment the personal care concentrate composition of the present invention comprises:
from 5 to 20 wt %, preferably from 10 to 15 wt % of an anionic surfactant selected from one or more of sodium lauroyl methyl isethionate, sodium cocoyl methyl isethionate and sodium oleoyl methyl isethionate;
from 5 to 20 wt %, preferably from 10 to 15 wt % of cocoamidopropyl betaine;
from 5 to 20 wt %, preferably from 10 to 15 wt % of an alkyl glucoside;
from 0.1 to 2 wt % of a biodegradable chelating agent;
from 0.1 to 5 wt %, preferably from 1.5 to 2.5 wt % of phenoxyethanol; and
from 0.1 to 5 wt %, preferably from 1.5 to 2.5 wt % of sodium benzoate.

The personal care concentrate composition of the present invention is preferably stable. By this we mean that the composition is suitably provided in a chemically and physically stable form.

Thus the composition suitably does not decompose on storage and is chemically stable under the conditions of light, heat and pressure at which it is prepared, used and stored.

The composition is suitably provided in a physically stable form. For example it does not change phase or separate into different phases on standing and is physically stable under the conditions of light, heat and pressure at which it is prepared, stored and used. For example a precipitate does not form from the composition and emulsions do not split.

Preferably the personal care concentrate compositions of the first aspect of the present invention remain stable on storage at 5° C. for 12 weeks.

Preferably the personal care concentrate compositions of the first aspect of the present invention remain stable on storage at 20° C. for 12 weeks.

Preferably the personal care concentrate compositions of the first aspect of the present invention remain stable on storage at 45° C. for 12 weeks.

Preferably the personal care concentrate composition of the first aspect of the present invention is a flowable composition. By flowable it is meant that the composition can be pumped or made to flow. The composition of the present invention may be any type of composition which can flow including free flowing compositions which can be easily poured.

The personal care concentrate composition of the present invention may be provided in any suitable form. Preferably it is of substantially uniform consistency. The composition may be in the form of an emulsion. However in preferred embodiments the composition is substantially homogeneous and is present as a single phase composition.

The viscosity of the personal care concentrate compositions of the present invention increases upon dilution with water.

Preferably the personal care concentrate composition increases in viscosity upon dilution with 0.1 to 20 parts water (by weight), preferably upon dilution with 0.5 to 15 parts water (by weight), more preferably upon dilution with 1 to 10 parts water (by weight), preferably upon dilution with from 1.5 to 6 parts water (by weight), more preferably 2 to 4 parts water (by weight).

The personal care concentrate composition of the first aspect of the present invention preferably has an initial viscosity at 20° C. of less than 2500 cP, preferably less than 2000 cP, for example from 100 to 2000 cP.

Upon dilution with 2 to 4 parts water (by weight) the personal care concentrate composition provides a ready-to-use personal care composition having a viscosity at 20° C. of at least 4000 cP, preferably at least 5000 cP, for example at least 6000 cP.

Viscosity may be measured by any suitable means and such means will be known to the person skilled in the art. For example the Brookfield DV-E Viscometer (Brookfield Engineering Laboratories, USA) may be used.

According to a second aspect of the present invention, there is provided a method of preparing a ready-to-use personal care composition, the method comprising providing a personal care concentrate composition of the first aspect and admixing said concentrate composition with water.

According to a third aspect of the present invention there is provided a ready-to-use personal care composition prepared by the method of the second aspect.

Preferred features of the second and third aspects are as defined in relation to the first aspect.

In the method of the second aspect the personal care concentrate composition is preferably mixed with from 0.1 to 20 parts water (by weight), preferably from 0.5 to 15 parts water (by weight), more preferably from 1 to 10 parts water (by weight), preferably from 1.5 to 6 parts water (by weight), more preferably from 2 to 4 parts water (by weight).

Preferably the method of the second aspect leads to an increase in the viscosity of the composition.

Preferably the method of the second aspect of the present invention provides a ready-to-use personal care composition in the form of a stable homogenous composition having an increased viscosity compared with the concentrate composition.

Suitably the method of the second aspect is carried out at ambient temperature. Suitably a stable homogenous composition may be prepared by admixture of the concentrate composition with water at ambient temperature with minimal stirring or agitation.

In preferred embodiments in the method of the second aspect the ready-to-use personal care composition is provided directly upon admixture of the personal care concentrate composition.

Preferably no additional salt is added to the concentrate composition when preparing the ready-to-use personal care composition.

The ready-to-use composition provided by dilution of the concentrate composition is a personal care composition. It may be any type of personal care composition, for example a shampoo, conditioner, cleanser, body wash, shower gel or moisturiser.

Preferably the ready-to-use composition provided by dilution of the concentrate composition is a “rinse-off” personal care composition. By this we mean to refer to a composition that is applied to the skin and/or hair and then removed by washing with water.

Preferably the ready-to-use composition is a shampoo or body wash.

Suitable the ready-to-use composition provided by the present invention is similar in appearance and consistency as a traditional personal care composition sold in fully formulated form. Suitably the ready-to-use composition provided by the invention has acceptable properties and characteristics and is accepted by the user as an equivalent product.

Preferably the ready-to-use composition has acceptable foaming properties.

Suitably the ready-to-use composition is stable. Thus the composition suitably does not decompose on storage and is chemically stable under the conditions of light, heat and pressure at which it is prepared, used and stored. Suitable the ready-to-use composition does not change phase or separate into different phases on standing and is physically stable under the conditions of light, heat and pressure at which it is prepared, stored and used. For example a precipitate does not form from the composition.

In some embodiments the concentrate composition may be provided in unit dose form. However in preferred embodiments the concentrate composition is provided in a size intended to provide a portion of ready-to-use composition suitable for multiple uses.

The personal care concentrate composition of the present invention is intended to be diluted by an end user to provide a ready-to-use composition personal care composition.

According to a fourth aspect of the present invention there is a provided a packaged personal care product comprising:
(i) a personal care concentrate composition comprising:
   (a) an anionic surfactant;
   (b) an amphoteric surfactant;
   (c) a non-ionic surfactant;
wherein the viscosity of the composition increases upon dilution with water; and
(ii) instructions for dilution of the personal care concentrate composition to provide a ready to-use personal care composition.

In some embodiments the packaged product may further comprise:
(iii) a vessel for dilution and/or storage.

Preferred features of the fifth aspect are as defined in relation to the first, second and third aspects.

In some embodiments the packaged personal care product may comprise multiple components and thus components of the concentrate composition may be packaged separately. However in preferred embodiments all components of the concentrate composition are provided in a single container and only water is added to provide the ready-to-use personal care composition.

The packaged personal care product may comprise a vessel for dilution and/or storage. The vessel preferably includes a mark to indicate to the use a suitable amount of water to add to the concentrate composition. In some embodiments the vessel may include two or more markings to indicate levels of water and concentrate composition that should be added.

In preferred embodiments all of the concentrate composition in a packaged product is diluted with water.

Preferably the vessel is suitable for diluting the concentrate composition and storing the ready-to-use composition. Preferably the vessel is provided with a lid.

In preferred embodiments the method of the second aspect is carried out in the vessel and involves pouring the concentrate composition into the vessel, adding water in an amount indicated on the vessel, closing the vessel and briefly shaking the vessel to ensure the contents are well mixed. The concentrate composition and the water may be added in any order.

Suitably the water and the concentrate composition do not completely fill the vessel to facilitate mixing upon gentle shaking.

The vessel is preferably re-usable. Thus once all of the ready-to-use composition has been consumed the vessel can be refilled from a new package of concentrate composition.

Thus the present invention may provide a “starter pack” packaged personal care product comprising components (i), (ii) and (iii) and a “refill pack” packaged personal care product comprising only components (i) and (ii).

According to a fifth aspect of the present invention there is provided a method of preparing personal care concentrate composition of the first aspect, the method comprising admixing:
(a) an anionic surfactant;
(b) an amphoteric surfactant; and
(c) a non-ionic surfactant.

Preferred features of the fifth aspect are as defined in relation to the first aspect.

Preferably the method of the fifth aspect does not involve the addition of sodium chloride.

Although residual sodium chloride may be present in components (a), (b) and/or (c), additional sodium chloride is preferably not added.

Preferably the method of the fifth aspect does not involve the addition of a free fatty acid.

Although residual free fatty acid may be present in components (a), (b) and/or (c), additional fatty acid is preferably not added.

In some preferred embodiments the composition of the first aspect is prepared according to the method of the fifth aspect.

In one embodiment the present invention provides a concentrate composition of the first aspect that is prepared without the addition of sodium chloride.

In one embodiment the present invention provides a concentrate composition of the first aspect that is prepared without the addition of a free fatty acid.

In one embodiment the present invention provides a concentrate composition of the first aspect that is prepared without the addition of a free fatty acid or sodium chloride.

The invention will now be further defined with reference to the following non-limiting examples.

### EXAMPLE 1

A personal care concentrated composition was prepared comprising the components listed in table 1:

TABLE 1

| Ingredient | Wt % active |
|---|---|
| Lauryl glucoside | 11.6 |
| Cocamido propyl betaine | 11.6 |
| Sodium lauroyl methyl isethionate | 11.6 |
| Sodium benzoate | 2 |
| Trisodium ethylene diamine disuccinate | 0.52 |
| Phenoxyethanol | 2 |
| Citric acid | To pH 6-6.5 |
| Water | balance |

Lauryl glucoside was provided as an aqueous solution comprising 51.2 wt % active surfactant-commercially available.

Cocamido propyl betaine was provided as an aqueous solution comprising 33.96 wt % active surfactant—commercially available.

Sodium lauroyl methyl isethionate was provided as a solid comprising 84.1 wt % active-commercially available.

Sodium benzoate was provided as the neat solid (100%).

Trisodium ethylene diamine disuccinate was provided as an aqueous solution comprising 30 wt % active—commercially available.

Phenoxyethanol was provided as the neat liquid (100%).

Citric acid was used as a 50% solution in water.

Sodium benzoate was added to water and mixed using a Eurostar 40 overhead stirrer (IKA Labortechnik, Germany) with a 4 blade PTFE 50 mm propeller (Fisher Scientific) at 150-200 rpm until clear. Lauryl glucoside and cocamido propyl betaine were added sequentially one at a time and stirred until a homogenous mixture was obtained. The formulation was then heated to 75° C. on an RCT basic heating plate (IKA Labortechnik, Germany). Once the temperature was reached, the trisodium ethylene diamine disuccinate was added, followed by the sodium lauroyl methyl isethionate in aliquots to prevent clumping and foaming, and stirred at approximately 500 rpm. When homogenous, the formulation was removed from the heat. Once the mixture had cooled to below 40° C., phenoxyethanol was added and mixed until homogenous. The pH was then adjusted with citric acid to reach a pH between 6 and 6.5.

### Example 2

A ready-to-use shampoo composition was prepared by combining 25% (w/w) of the prepared concentrate of example 1 and 75% (w/w) of water, which were manually shaken for 15-20 seconds, then left to rest to deaerate.

### Example 3

The Brookfield DV-E Viscometer (Brookfield Engineering Laboratories, USA) was used to take measurements at 20 rpm with spindle 5, at 20° C. for the compositions of example 1 and example 2. The results are in table 2.

TABLE 2

| Composition | Viscosity (cP) |
|---|---|
| Example 1 - concentrate | 1980 |
| Example 2 - ready-to-use | 7580 |

These results show that the composition of Example 1 can be diluted to give a thickened composition without the need for the additional of agents such as NaCl or polymer viscosifiers.

### Example 4

A further concentrate composition of the invention was prepared by adding 4 wt % of a conditioning agent to the composition of example 1. The conditioning agent chosen contained 20 wt % of laurdimonium hydroxypropyl hydrolyzed keratin and was sold under trade mark Kerestore 2.0. This composition was then diluted with water as described in example 2.

### Example 5

The composition of example 1 was stored at 5° C., 20° C. and 45° C. for 12 weeks. All compositions remained stable throughout.

1. A personal care concentrate composition comprising:
(a) an anionic surfactant;
(b) an amphoteric surfactant; and
(c) a non-ionic surfactant;
wherein the viscosity of the composition increases upon dilution with water.

2. The personal care concentrate composition according to claim 1 wherein component (a) comprises at least one acyl alkyl isethionate surfactant of formula (I): 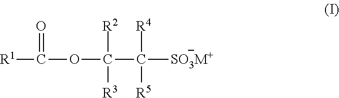
wherein R¹ represents an optionally substituted C₄-C₃₆ hydrocarbyl group;
each of R², R³, R⁴ and R⁵ independently represents hydrogen or a C₁-C₄ alkyl group and
wherein at least one of R¹, R³, R⁴ and R⁵ is not hydrogen; and M^{t} represents a cation

3. The personal care concentrate composition according to claim 2, wherein the acyl alkyl isethionate surfactant of formula (1) is a compound of formula R¹ COOCHR²CHR⁴SO₃⁻ M⁺ in which one of R² and R⁴ is methyl and the other is hydrogen, preferably comprising approximately 90% of compounds in which R² is methyl and R⁴ is hydrogen and approximately 10% of compounds in which R² is hydrogen and R⁴ is methyl.

4. The personal care concentrate composition according to claim 1, wherein component (b) comprises a betaine, preferably cocoamidopropyl betaine.

5. The personal care concentrate composition according to claim 1, wherein component (c) comprises an alkyl glucoside, preferably lauryl glucoside.

6. The personal care concentrate composition according to claim 1, which has a viscosity at 20° C. of less than 2500 cP.

7. The personal care concentrate composition according to claim 1 which upon dilution with 2 to 4 parts water (by weight) provides a ready-to-use personal care composition having a viscosity at 20° C. of at least 5000 cP.

8. The personal care concentrate composition according to claim 1, wherein the weight ratio of component (a) to component (b) to component (c) is 1:1±0.5:1±0.5.

9. The personal care concentrate composition according to claim 1 which comprises from 5 to 20 wt % component (a): from 5 to 20 wt %, component (b); and
from 5 to 20 wt % component (c).

10. The personal care concentrate composition according to claim 1, wherein component (a), component (b) and component (c) together comprise at least 30 wt % of the personal care concentrate composition.

11. The personal care concentrate composition according to claim 1, which comprises:
from 5 to 20 wt % of an anionic surfactant;
from 5 to 20 wt % of an amphoteric surfactant;
from 5 to 20 wt % of a non-ionic surfactant;
from 0.1 to 2 wt % of a chelating agent; and
from 0.1 to 10 wt % of one or more preservatives and/or antioxidants.

12. The personal care concentrate composition according to claim 1 which comprises:
from 5 to 20 wt % of an anionic surfactant selected from one or more of sodium lauroyl methyl isethionate, sodium cocoyl methyl isethionate and sodium oleoyl methyl isethionate;
from 5 to 20 wt % of cocoamidopropyl betaine;
from 5 to 20 wt % of an alkyl glucoside;
from 0.1 to 2 wt % of a biodegradable chelating agent;
from 0.1 to 5 wt % of phenoxyethanol; and
from 0.1 to 5 wt % of sodium benzoate.

13. A method of preparing a ready-to-use personal care composition, the method comprising providing a personal care concentrate composition as claimed in claim 1 and admixing said concentrate composition with water.

14. A ready-to-use personal care composition prepared by the method of claim 13.

15. A packaged personal care product comprising:
(i) a personal care concentrate composition comprising:
(a) an anionic surfactant;
(b) an amphoteric surfactant;
(c) a non-ionic surfactant;
wherein the viscosity of the composition increases upon dilution with water; and
(ii) instructions for dilution of the personal care concentrate composition to provide a ready to-use personal care composition.

16. The packaged personal care product according to claim 15 which further comprises:
(iii) a vessel for dilution and/or storage.
